(19) United States
(12) Patent Application Publication
(10) Pub. No.: US 2013/0245620 A1
(43) Pub. Date: Sep. 19, 2013

(54) REDUCED ADVANCEMENT FORCE NEEDLE AND METHODS OF MANUFACTURE

(71) Applicants: Anthony C. ARRIGO, North Andover, MA (US); Ho-Youn MOON, Hwasung-shi (KR)

(72) Inventors: Anthony C. ARRIGO, North Andover, MA (US); Ho-Youn MOON, Hwasung-shi (KR)

(21) Appl. No.: 13/763,289

(22) Filed: Feb. 8, 2013

Related U.S. Application Data
(60) Provisional application No. 61/597,517, filed on Feb. 10, 2012.

(51) Int. Cl. A61B 18/18; B29C 63/22

(52) U.S. Cl. CPC A61B 18/18; B29C 63/22
USPC 606/33; 156/221

(57) ABSTRACT

A needle apparatus and method of manufacturing the same arc disclosed. The needle apparatus includes a tubular needle component having a handling end including a needle hub and an insertion end including a pointed portion configured for piercing tissue of a patient. The needle apparatus also includes a cover forming a sleeve surrounding the tubular needle component. The cover extends along an outer surface of the tubular needle component such that the insertion tip of the needle remains uncovered. The cover includes a tapered end coupled to the tubular needle component adjacent to the insertion tip of the needle. The tapered end of the cover has a taper angle of 5-20 degrees with respect to the outer surface of the tubular needle component.

### CROSS REFERENCE TO RELATED APPLICATIONS

The present application claims priority to U.S. Provisional Application No. 61/597,517, filed Feb. 10, 2012, entitled “REDUCED ADVANCEMENT FORCE NEEDLE,” which application is incorporated herein by reference in its entirety.

### TECHNICAL FIELD

The present embodiments relate generally to a needle apparatus and methods of making a needle apparatus.

### BACKGROUND

During surgical procedures on the vertebrae and other parts of a patient's body, a needle, such as a radiofrequency needle, may be used. As it relates to the vertebrae, a radiofrequency needle may be used to selectively destroy nerves that carry pain impulses. The radiofrequency needle generates electrical energy that when applied to a nerve(s), such as nerve(s) of the superior hypogastrid plexus, creates a lesion on the nerve, thereby damaging (i.e. ablating) the nerve that carries the pain impulses. As it relates to the heart, a radiofrequency needle may also be used to ablate abnormal electrical pathways in heart tissue that cause atrial fibrillation.

A radiofrequency needle typically includes a cannula. A coating (e.g. insulation) covers a majority of the cannula of a radiofrequency needle so that the electrical energy transmitted through the radiofrequency needle passes only into the surrounding tissue from the exposed or uncovered tip of the cannula in close proximity to the tissue. There is a severe junction between a portion of the cannula covered with the protective coating and a portion of the cannula that is not covered with the protective coating, which creates an uneven and abrupt transition between the covered and uncovered portions of the needle.

As shown in FIGS. 1-4, disadvantages result when a conventional radiofrequency needle 101 is introduced into a patient's body. The severe junction 120 between the portion of the cannula 102 covered with a coating 103 and the portion not covered with the coating 103 leads to an increased penetration force experienced by patients when the junction between the covered and uncovered portion of the needle enters a patient. The severe junction 120 may also lead to snagging and/or retracting of the covering 103. The snagging and/or retracting increases the amount of the cannula that is uncovered, thereby increasing the likelihood that the needle will apply electrical energy to unintended parts of the body and subsequently heat and damage a larger area of the body than desired. The unintended heating may damage healthy nerves, may cause bleeding, or may lead to other detrimental effects. The snagging may also lead to portions of the coating falling off from the cannula and entering into a patient's body as a contaminant.

### SUMMARY

The inventors have appreciated that inventive embodiments disclosed herein provide a needle requiring reduced advancement forces to securely penetrate the needle into a patient's body.

Accordingly, various exemplary embodiments provide a reduced advancement force needle via a needle apparatus that includes a tubular needle component and a cover forming a sleeve surrounding the tubular needle component. The tubular needle component includes a handling end having a needle hub and an insertion end having a pointed portion configured for piercing tissue of a patient. The cover extends along an outer surface of the tubular needle component such that the insertion tip of the needle remains uncovered. The cover includes a tapered end coupled to the tubular needle component adjacent to the insertion tip of the needle. The tapered end of the cover has a taper angle of 5-20 degrees with respect to the outer surface of the tubular needle component.

The tubular needle component is configured to conduct electricity in accordance with various embodiments.

The tubular needle component may be coupled to an electrical heating clement. The electrical heating element may include a radiofrequency electrode,

In accordance with various embodiments, the cover of the needle apparatus is formed of an electrically insulating material. The cover may be composed of polyester in accordance with some embodiments. The polyester may include heat shrinkable tubing. The cover may be composed of polypropylene in accordance with some embodiments.

In accordance with some embodiments, the tubular needle component is hollow.

The cover may he crimped to the ubular needle component in accordance with various embodiments.

Various exemplary embodiments provide a method of producing a needle apparatus that include coupling a cover forming a sleeve to a tubular needle component. The tubular needle component includes a handling end having a needle hub and an insertion end having a pointed portion configured for piercing tissue of a patient. The cover is coupled to the tubular needle component such that the cover extends along an outer surface of the tubular needle component such that the insertion tip of the needle remains uncovered. The method further includes forming an end of cover adjacent to the insertion tip of the needle into a tapered end having a taper angle of 5-20 degrees with respect to the outer surface of the tubular needle component.

In accordance with various embodiments, the cover is coupled to the tubular needle component via a heating source. The end of the cover adjacent to the insertion tip of the needle may be formed into a tapered end having a taper angle of 5-20 degrees via a mold. The mold may form the cover into a tapered end after heat is applied by the heating source.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a side view of a portion of a first conventional needle.

FIG. 2 is a side view of a portion of a second conventional needle.

FIG. 3 is a side view of a portion of a third conventional needle.

FIG. 4 is a side view of a portion of a fourth conventional needle.

FIG. 5 is a side view of a portion of a needle in accordance with an exemplary inventive embodiment.

FIG. 6 is a side view of a portion of a needle with a coating at a first angle in accordance with an exemplary inventive embodiment.

FIG. 7 is a side view of a portion of a needle with a coating at a second angle in accordance with an exemplary inventive embodiment.

FIG. 8a is a side view of a portion of a needle when the covering first couples to the cannula.

FIG. 8b is a side view of the needle of FIG. 8a after the covering is coupled to the cannula and an angle is formed on the coating in accordance with an exemplary inventive embodiment.

FIG. 9 is a side view of a forming mechanism for forming an angle on a coating of a needled in accordance with an exemplary inventive embodiment.

FIG. 10 is a side view of a needle with a coating where the needle includes a curved portion in accordance with an exemplary inventive embodiment.

FIG. 11 is a side view of a needle with a coating where the needle is substantially straight in accordance with an exemplary inventive embodiment.

FIG. 12 is a graph showing load results for a conventional needle.

FIG. 13 is a side view of a conventional needle after being inserted into a medium.

FIG. 14 is a graph showing load results for a needle in accordance with an exemplary inventive embodiment disclosed herein.

FIG. 15 is a chart comparing penetration force results for conventional needles to needles formed in accordance with exemplary inventive embodiments of the present disclosure.

FIG. 16 is a graph showing the penetration force results for a first needle in accordance with an exemplary inventive embodiment of the present disclosure.

FIG. 17 is a graph showing the penetration force results for a second needle in accordance with an exemplary inventive embodiment of the present disclosure.

FIG. 18 is a graph showing the penetration force results for a third needle in accordance with an exemplary inventive embodiment of the present disclosure.

FIG. 19 is a graph showing the penetration force results for a first conventional needle.

FIG. 20 is a graph showing the penetration force results for a second conventional needle.

FIG. 21 is a graph showing the penetration force results for a third conventional needle.

The features and advantages of the inventive concepts disclosed herein will become more apparent from the detailed description set forth below when taken in conjunction with the drawings.

### DETAILED DESCRIPTION

Following below are more detailed descriptions of various concepts related to, and embodiments of inventive apparatuses and methods for manufacturing and using a reduced advancement force needle. While the disclosure specifically discusses a radiofrequency reduced advancement force needle, the reduced advancement force needle according to inventive embodiments disclosed herein may pertain to needles other than radiofrequency needles, such as any needle that is configured to ablate a part of the body or otherwise includes a covering. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

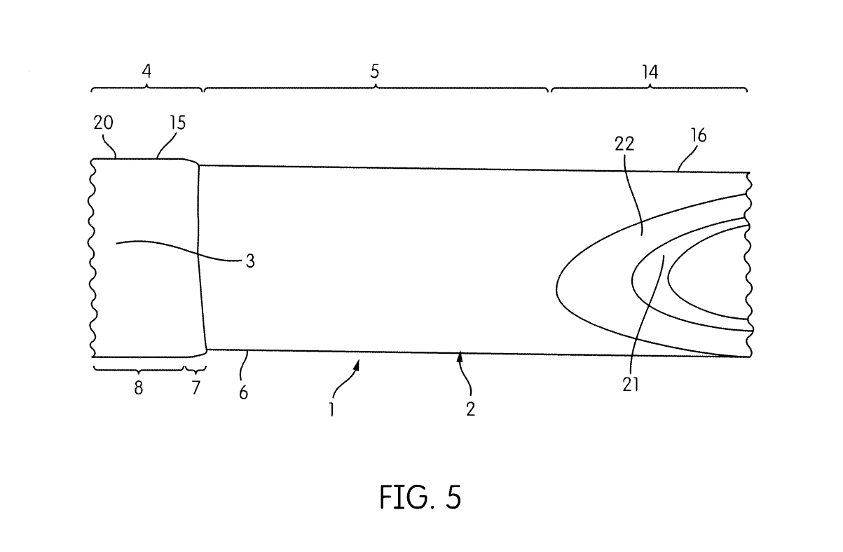

FIGS. 5-11 illustrate a needle 1 for ablating a part of the body in accordance with exemplary inventive embodiments. The needle 1 may include a tubular needle component such as tube 2, depicted as a hollow tube (e.g. cannula) in the illustrated embodiment, and a cover or covering 3 coupled to the hollow tube 2 and forming a sleeve surrounding hollow tube 2. The hollow tube 2 may be any suitable size. For example, the hollow tube 2 (e.g. cannula) may be a 18-22 gauge needle. The hollow tube 2 may include a handling end such as hollow tube first portion 4 (e.g. a cannula first portion) and an insertion end such as hollow tube second portion 5 wherein the insertion end of the needle is positioned (e.g. a cannula second portion). The hollow tube second portion 5 may extend From the hollow tube first portion 4. The hollow tube first portion 4 and hollow tube second portion 5 may have a common hollow outer tube surface 6 that extends along a substantial length of the hollow tube 2.

The first and second hollow tube portions 4, 5 may be made of any suitable material that is configured to transfer electrical energy. For example, the first and second hollow tube portions 4, 5 may comprise a 304, 304L, 316, or 316L stainless steel hypodermic tube or any other suitable material used to manufacture a needle, such as nitinol. The first and second hollow tube portions 4, 5 may comprise the same or different materials. Preferably, the first and second hollow tube portions 4, 5, comprise the same material and are integral.

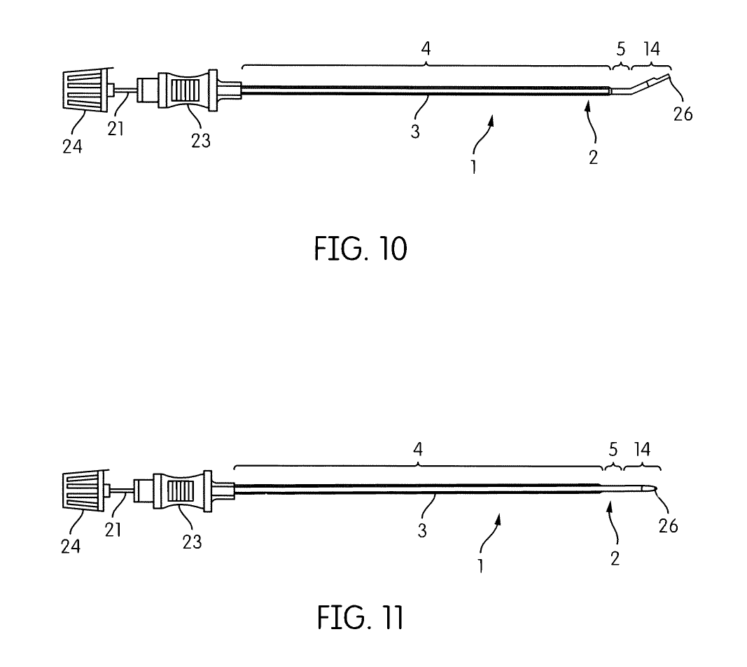

The hollow tube 2 may also include a tip portion 14 (e.g. cannula tip portion) that extends from the second hollow tube portion 5 and is distal from the first hollow tube portion 4 (FIG. 5). The tip portion may begin in a pointed geometry configured to penetrate the tissue of a patient. The tip portion 14 may be curved (FIG. 10) or straight (FIG. 11) such that the tip portion 14 does not or does extend along the same axis as the hollow tube first portion 1.

The tip portion 14 may comprise any suitable material that is configured to transfer electrical energy. For example, the tip portion 14 may comprise a 304, 304L, 316, 316L stainless steel hypodermic tube or any other suitable material used to manufacture a needle, such as nitinol. The tip portion 14 may comprise the same material or a different material from the first hollow tip portion 4 and/or second hollow tube portion 5. Preferably, the tip portion 14 comprises the same material as the first and second hollow tube portions 4, 5 and is integral with the first and second hollow tube portions 4, 5.

The tip portion 14 may include an opening 22 configured to receive a heating element 21. The heating element 21 may be any element such as an electrical heating element that may be electrically stimulated and may transfer electrical energy to the hollow tube 2 for heating and ablating a part of the body contacted thereby. For example, the heating element 21 may comprise a radiofrequency electrode, radiofrequency generator or stylet. The heating element 21 may comprise any suitable material. For example, the heating element 21 may comprise 304 stainless steel.

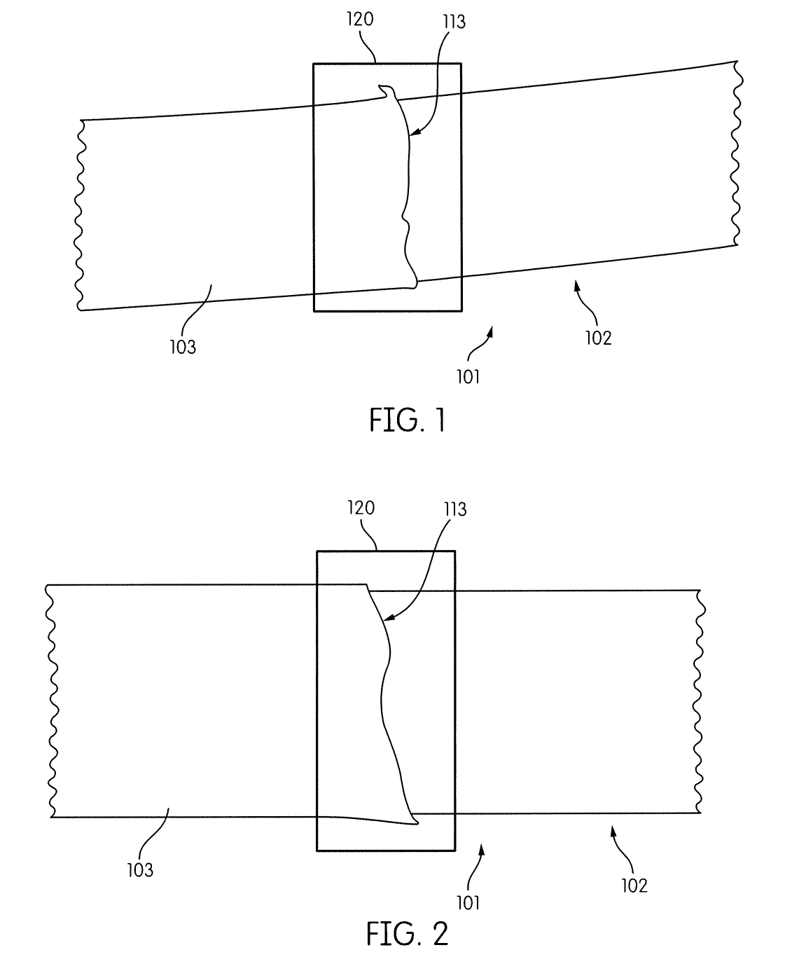

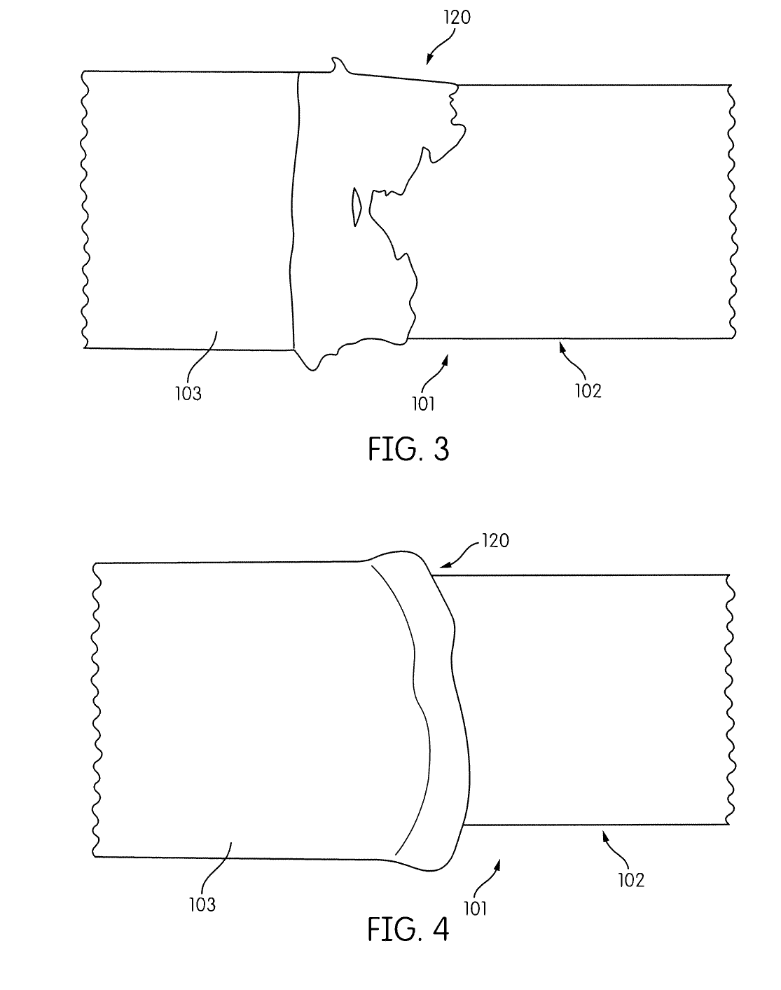

The heating element 21 may include a cap 24 and may insert through a needle hub 23 of the hollow tube 2 (FIGS. 10-1 1). The cap 24 may be made of any suitable material, such as for example plastic (e.g. polypropylene), chrome or nickel plated brass. The hub 23 may be made of any suitable material, such as for example plastic (e.g. all resin types, including but not limited to k-resin), chrome or nickel plated brass. The heating element 21 and cap 24 may be coupled together by any suitable material, such as UV curable adhesive. Similarly, the hollow tube 2 and the hub 23 may be coupled together by any suitable material, such as UV curable adhesive.

The covering 3 may be coupled to the hollow tube outer surface 6 and may extend along the hollow tube first portion 4 (FIGS. 5-7). For example, the covering may be crimped onto surface 6, may be bonded via heating or other means, or may be coupled via a friction fit formed by heat shrinking covering 3 to the outer surface 6. The covering 3 does not extend past the hollow tube first portion 4 to the hollow tube second portion 5. Consequently, the covering 3 only extends along the hollow tube first portion 4.

The covering 3 protects parts of a patient's body (e.g. tissue) that surround the hollow tube 2 when the hollow tube 2 enters the body. The covering 3 ensures that electrical energy does not pass from the hollow tube 2 into an undesired part of the body. The covering 3 may be made of any material that is capable of ensuring that electric current does not pass through the covering into a part of the body. For example, the covering 3 (e.g. insulation) may comprise polyester, (such as black polyester heat shrink tubing), polypropylene, or other electrically insulating materials.

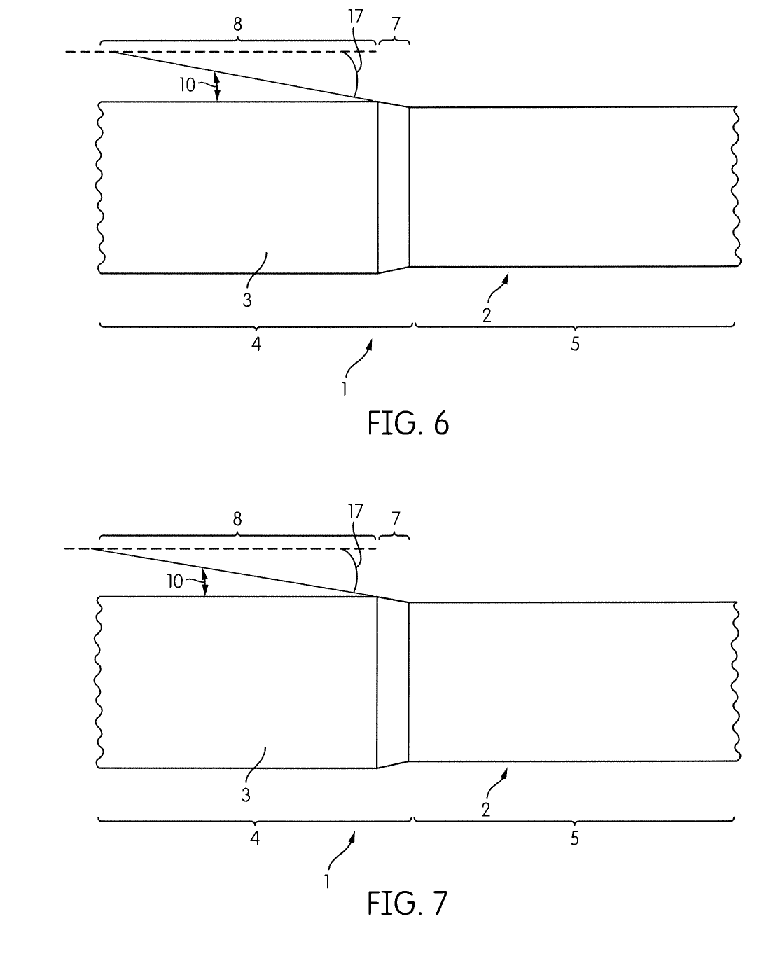

The covering 3 may include a covering junction 7 (FIGS. 5-7). The covering junction 7 may abut the hollow tube second portion 5 and be distal from the tip portion 14. The covering junction 7 may be tapered to form a tapered end at a taper angle or covering junction angle 10 of between approximately 5°-20° relative to the hollow tube outer surface 6 and, more preferably, between approximately 9°-10.2° relative to the hollow tube outer surface 6. For example, as shown in FIGS. 6-7, the covering junction angle 10 may be approximately 9° (FIG. 6) or approximately 10.2° (FIG. 7). While the covering junction 7 is shown in the preferred embodiments as having a continuous covering junction angle 10 extending to the intersection with a covering first portion 8 (described below), it is understood that a differently tapered portion, an arced portion, or other shaped portion may be disposed between the covering junction 7 and the covering first portion 8. In contrast to conventional needles, the covering junction angle 10 decreases the likelihood that the covering 3 will snag and/or retract because the covering junction angle 10 provides a smooth and gradual transition from the hollow tube second portion 5 to the covering 3.

Tests were conducted to determine whether covering 3 of needle 1 and the covering of conventional needles retracted and/or snagged when inserted into different mediums. Three different mediums were used in the testing. Each of the mediums had a different hardness from the other two mediums. The first medium was the softest and comprised polyethylene. The third medium was the hardest and comprised rubber. When a needle 1 according to inventive embodiments of the present disclosure is inserted through each of the three mediums, covering 3 did not retract. In contrast, when conventional needles were inserted through the same mediums, some of the coverings retracted and/or snagged. All of the conventional needle coverings retracted and/or snagged when inserted through the third medium.

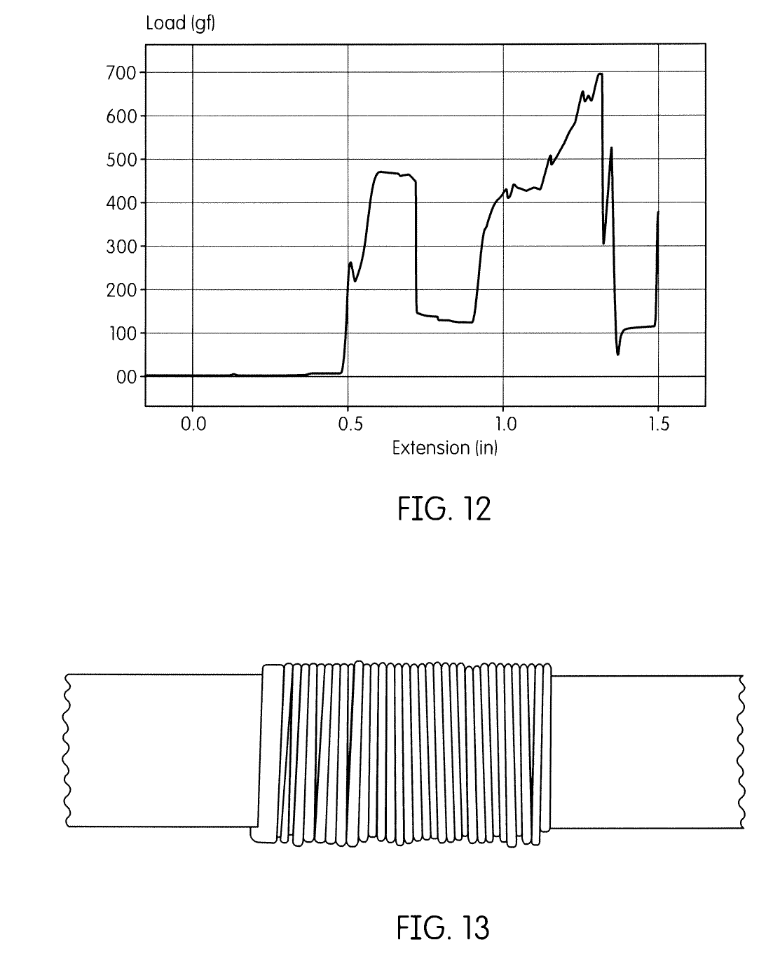

FIG. 12 shows the results for a conventional needle when inserted into the third medium. At approximately 0.6 inches the conventional needle enters the third medium. At approximately 0.9 inches the covering of the conventional needle enters the third medium. When the covering of the conventional needle enters the third medium, the amount of force exerted on the covering increases (see between approximately 0.9-1.4 inches in FIG. 12) due to the covering junction angle between the exposed part of the needle and the covering. As a result of the increased force caused by the junction angle, the covering retracts and/or snags to expose previously covered portions of a cannula (see FIG. 13).

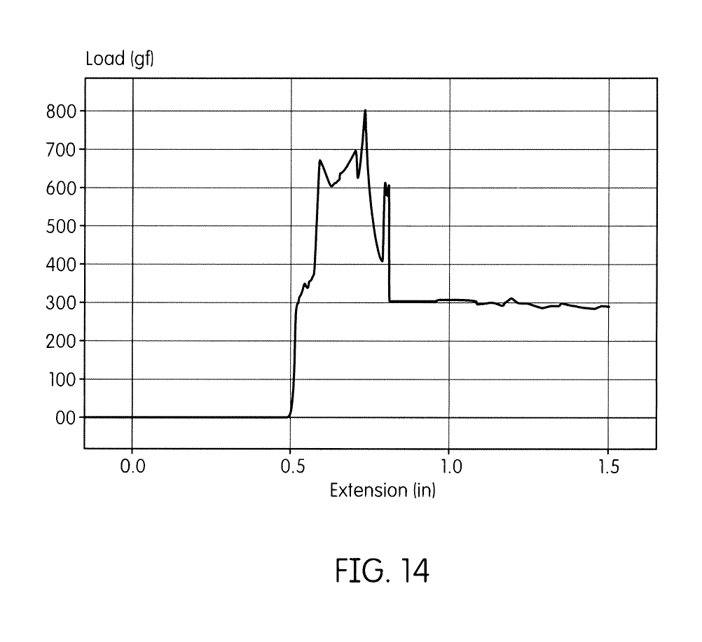

In contrast, FIG. 14 shows the results of a needle 1 according to the present invention when inserted into the third medium. At approximately 0.6 inches the needle 1 enters the third medium. At approximately 0.8 inches the covering 3 enters the third medium. When the covering 3 enters the third medium, the force exerted on the covering decreases and remains fairly constant due to the covering junction angle 10 (see approximately 0.8-1.5 inches in FIG. 14). The covering junction angle 10 helps eliminate undesired ablation (i.e. application of electrical energy that causes a part of the body to heat up, thereby ablating that part of the body) because the exposure of undesired parts of the hollow tube 2, due to snagging and/or retracting, does not occur. The exposure refers to areas of the hollow tube 2 that are intended to be coated and covered by the covering material, but become uncovered because of snagging and/or retracting of the covering material.

In contrast to conventional needles, the covering junction angle 10 also reduces the load and drag force (i.e. penetration force) exerted on a patient when the covering junction passes through tissue, thereby reducing (or eliminating) the ability of the covering to snag and/or retract at the covering junction. Conventional needles may include a covering junction angle 10 of as large as approximately 87°, 90° or 94°. As a result of the large covering junction angle of conventional needles, the covering may snag or retract due to the increased load and drag force exerted on the patient at the covering junction.

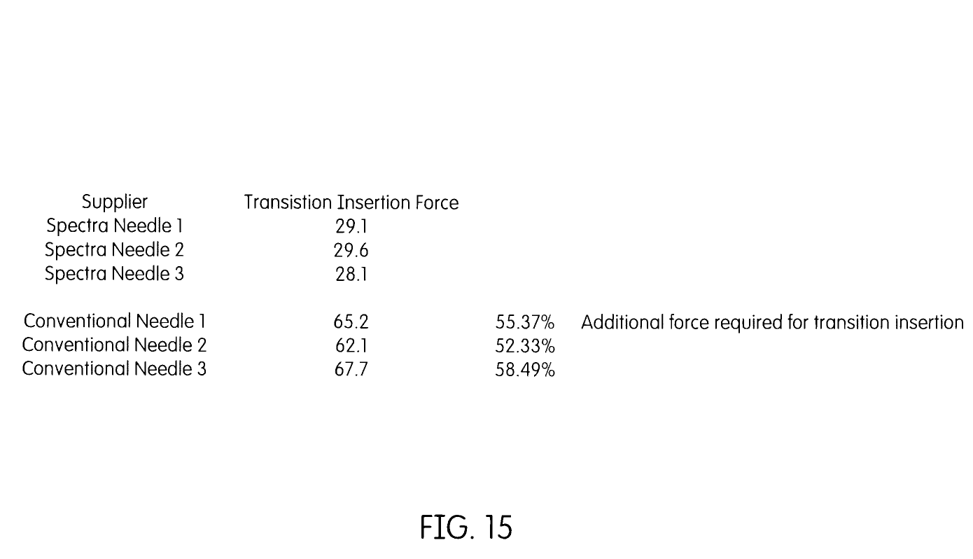

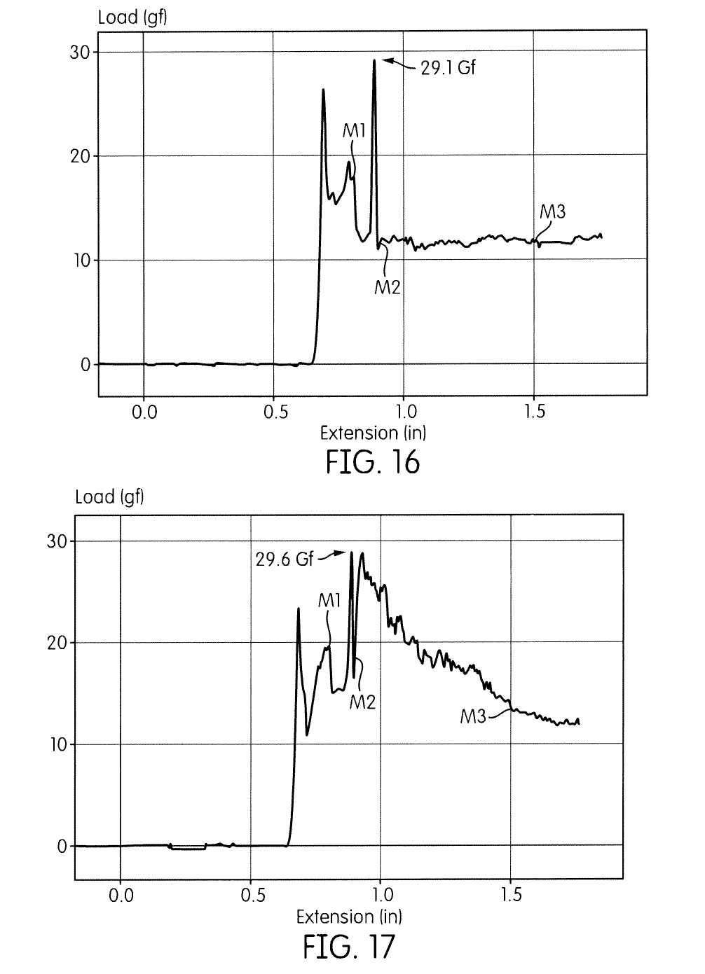

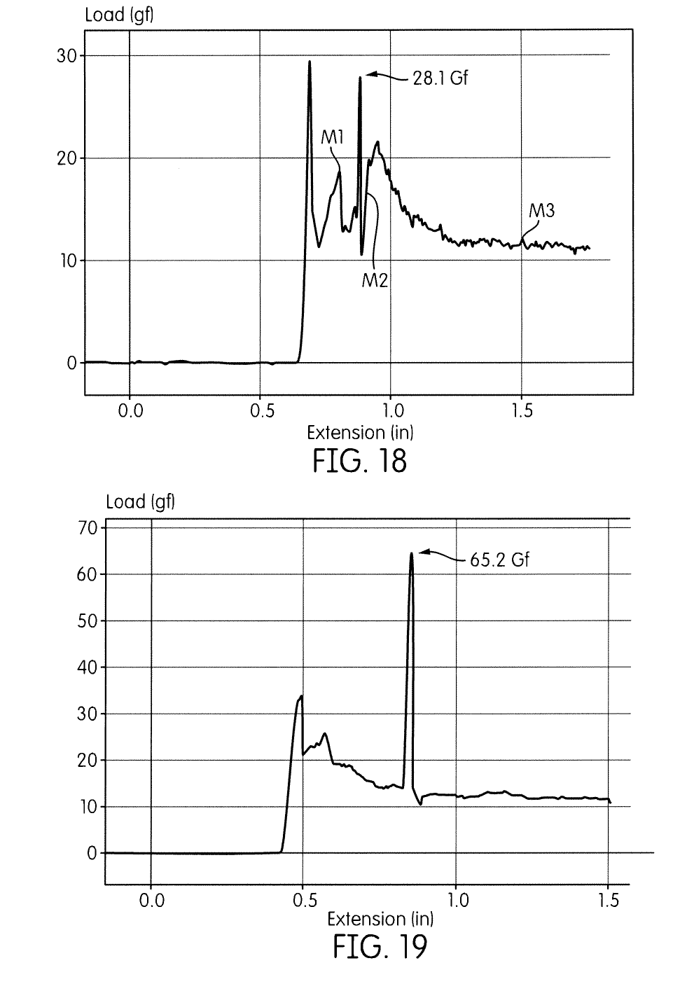

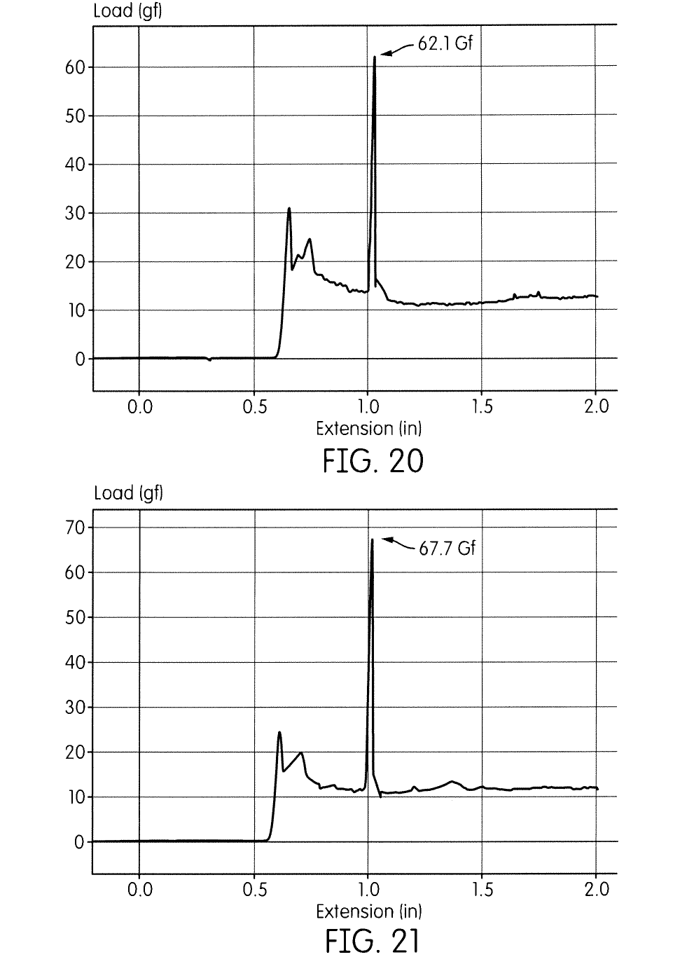

Needle 1 may reduce the penetration force on a patient with respect to conventional needles by as much about 60%. For example, as shown in FIG. 15, three needles 1, according to exemplary inventive embodiments of the present disclosure, were compared to three conventional needles. The three needles 1 each had a covering junction angle of approximately 9°. The penetration force of the needles 1 at the transition between the first and second hollow tube portions 4, 5 (approximately 0.9-1.0 inches from the end 26 of the needle 1, sec FIGS. 10-11 and 16-21) ranged from approximately 28.1 gf to 29.6 gf while the penetration force of the conventional needles at the same transition ranged from approximately 62.1 gf to 67.7 gf, thereby resulting in a reduction of penetration force exerted by approximately 52.33% to 58.49%. FIGS. 16-21 show graphs depicting the data shown in FIG. 15 for the three needles 1 and conventional needles.

The covering 3 may also include a covering first portion 8 that extends from the covering junction 7 and is more distal from the hollow tube second portion 5 than the covering junction 7 (FIG. 5). The outer surface 20 of the covering first portion 8 may be substantially parallel to the hollow tube outer surface 6 of the hollow tube 2 (i.e. the first portion 8 may be substantially parallel to the hollow tube 2). The thickness of covering 3 (i.e. the radial thickness of the sidewalls forming tubular covering 3) may he any suitable thickness. For example, the thickness may range from approximately 0.001-0.0005 inches. The covering first portion 8 may include a covering outer diameter 15 and the hollow tube outer surface 6 may include a hollow tube outer diameter 16 (e.g. cannula outer diameter) that is smaller than the covering outer diameter 15 (FIG. 5).

The covering first portion 8 may be at a covering first portion angle 17 (FIGS. 6-7) relative to the covering junction 11. The covering first portion angle 17 complements the covering junction angle 10. For example, if the covering junction angle 10 is approximately 9° then the covering first portion angle is approximately 81° (FIG. 6) and if the covering junction angle is approximately 10.2° then the covering first portion angle is approximately 79.8° (FIG. 7).

A method of making a needle 1, such as a needle to ablate a part of the body, may include surrounding the hollow tube first portion 4 of the hollow tube 2 with a covering 3. The hollow tube 2 may he surrounded by the covering 3 by placing (e.g. inserting the covering 3 over the hollow tube 2 by hand or by a machine) the covering 3 over the hollow tube 2. The method may also include surrounding the hollow tube second portion 5 and/or tip portion 14 with the covering 3. After surrounding one or more portions of the hollow tube 2 with the covering 3, the covering 3 is positioned to only surround the hollow tube first portion 4.

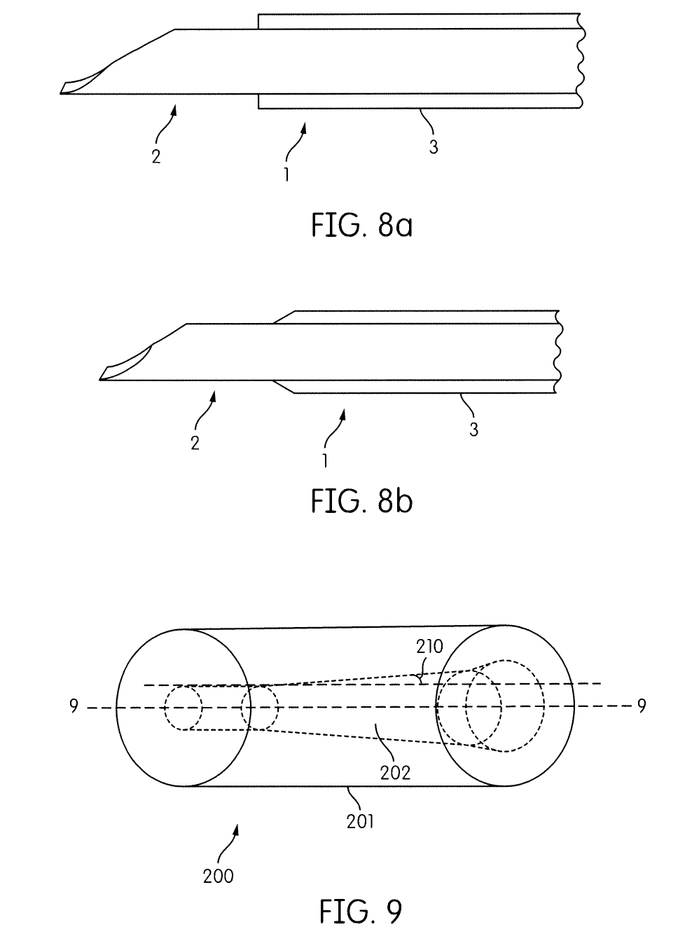

The method of making the needle 1 also includes applying heat to the covering 3 until the covering couples to only the hollow tube first portion 4 (i.e. heat shrinking the covering 3 to the hollow tube first portion 4). The covering 3 does not couple to the hollow tube second portion 5. The heat may be applied by any suitable mechanism, such as a heat press, that couples (i.e. heat shrinks) the covering 3 to the hollow tube first portion 4. The heat may be applied to the hollow tube second portion 5 and/or the tip portion 14 when the heat is applied to the hollow tube first portion 4. When the heat is applied to the hollow tube first portion 4, the outer diameter of the covering 3 gets smaller and forms a substantially uniform or uniform diameter around the needle 1 (FIG. 8).

After applying heat to the covering 3 such that the covering 3 is coupled to the hollow tube first portion 4, a forming mechanism 200 (e.g. forming tool, tool) may be used to apply heat to the covering junction 7 (i.e. heat shrinking the covering junction 7) until the covering junction 7 is at a covering junction angle 10 to the covering first portion 8 (FIG. 9). As previously discussed, the covering junction angle 10 may be between approximately 5°-20° from the hollow tube outer surface 6.

The forming mechanism 200 may be any suitable mechanism. As shown in FIG. 9, the forming mechanism 200 may include housing or mold 201 and an opening 202 that extends through the mold 201 along a lateral axis 9-9 of the forming mechanism 200. The mold 201 may comprise any suitable material. For example, the mold 201 may comprise steel, such as a D2 steel. The opening 202 is configured to receive the needle 1 to create the covering junction angle 10. Once the covering junction angle 10 is created, the needle 1 may be removed from the forming mechanism 200. Preferable, the needle 1 is placed in the forming mechanism 200 after heat applied to the covering 3 so the covering 3 forms a substantially uniform or uniform diameter around the needle 1.

The forming mechanism 200 includes a forming mechanism angle 210. The forming mechanism angle 210 is the same as the covering junction angle 10. When heat is applied to the needle 1, after the opening 202 of the foisting mechanism 200 receives the needle 1, the shape of the covering junction 7 changes to match the shape of the opening 202 of the forming mechanism 200. The change modifies the angle of the covering junction from substantially parallel or parallel to the hollow tube outer surface 6 to the covering junction angle 10.

The shape of the opening 202 may be frustoconically shaped. The changing diameter of the frustoconically shaped opening differs depending on the gauge of the needle 1, such that larger needles require a larger diameter opening. For example, 18 gauge needles require a larger diameter opening than 22 gauge needles.

Heat may be applied to the needle 1, after the opening 202 of the forming mechanism 200 receives the needle 1, by any suitable mechanism. For example, the forming mechanism 200 may contain heating elements or heating elements may be outside of the forming mechanism 200. If the forming mechanism 200 contains heating elements, the heating elements may be within the housing 201 or inside the opening 202. If the heating elements are inside the opening 202, the heating elements may be attached to one or more surfaces of the opening 202. If the heating elements are outside of the forming mechanism 200, the heating elements must be configured to transfer heat through the housing 201 to the opening 202 of the forming mechanism 200. Although the present disclosure discloses having a plurality of heating elements, only one heating element may be used to apply heat to the needle 1.

As utilized herein, the terms “approximately,” “about,” “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described without restricting the scope of these features to the precise numerical ranges provided. Accordingly, these terms should be interpreted as indicating that insubstantial or inconsequential modifications or alterations of the subject matter described and are considered to be within the scope of the disclosure.

It should be noted that the term “exemplary” as used herein to describe various embodiments is intended to indicate that such embodiments arc possible examples, representations, and/or illustrations of possible embodiments (and such term is not intended to connote that such embodiments are necessarily extraordinary or superlative examples).

For the purpose of this disclosure, the term “coupled” means the joining of two members directly or indirectly to one another. Such joining may be stationary or moveable in nature. Such joining may be achieved with the two members or the two members and any additional intermediate members being integrally formed as a single unitary body with one another or with the two members or the two members and any additional intermediate members being attached to one another. Such joining may be permanent in nature or may be removable or releasable in nature.

It should be noted that the orientation of various elements may differ according to other exemplary embodiments, and that such variations are intended to be encompassed by the present disclosure. It is recognized that features of the disclosed embodiments can be incorporated into other disclosed embodiments.

It is important to note that the constructions and arrangements of the reduced advancement force needle or components thereof as shown in the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter disclosed. For example, elements shown as integrally formed may be constructed of multiple parts or elements, the position of elements may be reversed or otherwise varied, and the nature or number of discrete elements or positions may be altered or varied. The order or sequence of any process or method steps may be varied or re-sequenced according to alternative embodiments. Other substitutions, modifications, changes and omissions may also be made in the design, operating conditions and arrangement of the various exemplary embodiments without departing from the scope of the present disclosure.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, describes techniques, or the like, this application controls.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or he able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, the technology described herein may be embodied as a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one clement of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of shall be closed or semi-closed transitional phrases, respectively, as set forth in the United States Patent Office Manual of Patent Examining Procedures, Section 2111.03.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

1. A needle apparatus comprising:
a tubular needle component having a handling end including a needle huh and an insertion end including a pointed portion configured for piercing tissue of a patient; and
a cover forming a sleeve surrounding the tubular needle component, the cover extending along an outer surface of the tubular needle component such that the insertion tip of the needle remains uncovered, the cover including a tapered end coupled to the tubular needle component adjacent to the insertion tip of the needle, the tapered end of the cover having a taper angle of 5-20 degrees with respect to the outer surface of the tubular needle component.

2. The apparatus of claim 1, wherein the tubular needle component is configured to conduct electricity.

3. The apparatus of claim 1, wherein the tubular needle component is coupled to an electrical heating element.

4. The apparatus of claim 3, wherein the electrical heating element is a radiofrequency electrode.

5. The apparatus of claim 1 wherein the cover is formed of an electrically insulating material.

6. The apparatus of claim 1, wherein the cover is composed of at least one of polyester and polypropylene.

7. The apparatus of claim 6, wherein the cover is composed of polyester that is heat shrinkable tubing.

8. The apparatus of claim 1, wherein the tapered end of the cover has a taper angle of 9-10.2 degrees with respect to the outer surface of the tubular needle component.

9. The apparatus of claim 1, wherein the tubular needle component is hollow.

10. The apparatus of claim 1, wherein the cover is crimped to the tubular needle component.

11. A method of forming a needle apparatus, the method comprising:
coupling a cover forming a sleeve to a tubular needle component, the tubular needle component having a handling end including a needle hub and an insertion end including a pointed portion configured for piercing tissue of a patient, the cover coupled to the tubular needle component such that the cover extends along an outer surface of the tubular needle component such that the insertion tip of the needle remains uncovered; and
forming an end of the cover positioned adjacent to the insertion tip of the needle into a tapered end having a taper angle of 5-20 degrees with respect to the outer surface of the tubular needle component.

12. The method of claim 11, wherein the cover is coupled to the tubular needle component via a heating source.

13. The method of claim 12, wherein the end of the cover adjacent to the insertion tip of the needle is formed into a tapered end having a taper angle of 5-20 degrees with a mold.

14. The method of claim 13, wherein the cover is formed into a tapered end with a mold after heat is applied from the heating source.

15. The method of claim 11, wherein the tubular needle component is configured to conduct electricity.

16. The method of claim 11, wherein the tubular needle component is configured to conduct electricity from a radiofrequency electrode.

17. The method of claim 11, wherein the cover is formed of an electrically insulating material.

18. The method of claim 11, wherein the cover is composed of polyester heat shrinkable tubing.

19. The method of claim 11, wherein the tapered end of the cover has a taper angle of 9-10.2 degrees with respect to the outer surface of the tubular needle component.

20. The method of claim 11, wherein the cover is coupled to the tubular needle component via a crimping mechanism,
